(19) United States
(12) Patent Application Publication
(10) Pub. No.: US 2025/0180733 A1
(43) Pub. Date: Jun. 5, 2025

(54) OCCUPANT POSITION ESTIMATING DEVICE AND OCCUPANT POSITION ESTIMATING METHOD

(71) Applicant: DENSO CORPORATION, Kariya-city (JP)

(72) Inventors: Shunsuke SHIBATA, Nisshin-city (JP); Daiki ITO, Nisshin-city (JP); Yuuji KAKUYA, Kariya-city (JP)

(21) Appl. No.: 19/051,035

(22) Filed: Feb. 11, 2025

Related U.S. Application Data
(63) Continuation of application No. PCT/JP2023/030169, filed on Aug. 22, 2023.

(30) Foreign Application Priority Data
Sep. 2, 2022 (JP) 2022-140167

(51) Int. Cl. G01S 13/88; G01S 7/41; G01S 13/87

(52) U.S. Cl. CPC G01S 13/88; G01S 7/412; G01S 13/87

(57) ABSTRACT

An occupant position estimating device includes a transmission control unit, a received strength obtaining unit, a separating unit, a headcount identifying unit, a distance identifying unit, and an occupant position estimating unit. The transmission control unit controls each of radars to transmit radio waves. The received strength obtaining unit obtains multiple received strengths of the radio waves that are received by the respective radars. The separating unit obtains separated waveforms for the occupants from waveforms each of which indicates a time variation of the multiple received strengths. The headcount identifying unit identifies a number of the occupants from the separated waveforms. The distance identifying unit identifies distances from each of the radars to the respective occupants based on a timing at which a human-specific fluctuation appears in the separated waveform. The occupant position estimating unit estimates a position of each of the occupants based on the distances.

### CROSS REFERENCE TO RELATED APPLICATIONS

The present application is a continuation application of International Patent Application No. PCT/JP2023/030169 filed on Aug. 22, 2023, which designated the U.S. and claims the benefit of priority from Japanese Patent Application No. 2022-140167 filed on Sep. 2, 2022. The entire disclosures of all the above applications are incorporated herein by reference.

### TECHNICAL FIELD

This disclosure relates to an occupant position estimating device and an occupant position estimating method for estimating the position of each of occupants in a vehicle cabin.

### BACKGROUND

All occupants in a vehicle cabin are required to wear seat belts in many countries. Accordingly, vehicle manufacturers are required to equip a system that warns the occupants who do not wear seat belts.

### SUMMARY

According to one aspect of the present disclosure, an occupant position estimating device to estimate a position of each of occupants in a vehicle cabin of a vehicle is provided. The occupant position estimating device includes a transmission control unit, a received strength obtaining unit, a separating unit, a headcount identifying unit, a distance identifying unit, and an occupant position estimating unit. The transmission control unit controls each of radars to transmit radio waves. The radars transmit and receive radio waves in the vehicle cabin. The received strength obtaining unit obtains multiple received strengths of the radio waves that are received by the respective radars. The separating unit obtains a separated waveform for each of the occupants through a process to isolate components corresponding respectively to the occupants from waveforms each of which indicates a time variation of the multiple received strengths that are received by the received strength obtaining unit. The headcount identifying unit identifies a number of the occupants in the vehicle cabin from the separated waveform of each of the occupants. The distance identifying unit identifies distances from each of the radars to the respective occupants in accordance with the number of the occupants identified by the headcount identifying unit based on a timing at which a human-specific fluctuation appears in the separated waveform. The occupant position estimating unit estimates a position of each of the occupants in the vehicle cabin based on the distances from each of the radars to the respective occupants. The received strength obtaining unit separately obtains reflection characteristic values that are received strengths of the radio waves that are transmitted by one of the radars and received by the one of the radars, and transmission characteristic values that are received strengths of the radio waves that are transmitted by one of the radars and received by another of the radars. The separating unit is configured to obtain the separated waveform for each of the occupants through a process to isolate the components corresponding respectively to the occupants from a waveform which indicates a time variation of the reflection characteristic values and a waveform which indicates a time variation of the transmission characteristic values. The headcount identifying unit identifies the number of the occupants in the vehicle cabin based on the separated waveform for each of the occupants.

According to another aspect of the present disclosure, an occupant position estimating device to estimate a position of each of occupants in a vehicle cabin of a vehicle is provided. The occupant position estimating device includes a transmission control unit, a received strength obtaining unit, a separating unit, a headcount identifying unit, a distance identifying unit, and an occupant position estimating unit. The transmission control unit controls each of radars to transmit radio waves. The radars transmit and receive radio waves in the vehicle cabin. The received strength obtaining unit obtains multiple received strengths of the radio waves that are received by the respective radars. The separating unit obtains a separated waveform for each of the occupants through a process to isolate components corresponding respectively to the occupants from waveforms each of which indicates a time variation of the multiple received strengths that are received by the received strength obtaining unit. The headcount identifying unit identifies a number of the occupants in the vehicle cabin from the separated waveform of each of the occupants. The distance identifying unit identifies distances from each of the radars to the respective occupants in accordance with the number of the occupants identified by the headcount identifying unit based on a timing at which a human-specific fluctuation appears in the separated waveform. The occupant position estimating unit estimates a position of each of the occupants in the vehicle cabin based on the distances from each of the radars to the respective occupants. At least one of the radars is a mobile terminal that is carried by one of the occupants. A position of the mobile terminal relative to the vehicle is identified by a system of the vehicle using a communication between the mobile terminal and at least three antennas disposed in the vehicle. The other of the radars is a radio wave detector that is disposed in the vehicle. A position of the radio wave detector relative to the vehicle is predetermined. The occupant position estimating unit is configured to estimate the position of each of the occupants in the vehicle cabin based on a distance from the position of the mobile terminal, which is identified by the system of the vehicle, to each of the occupants and a distance from the position of the at least one radio wave detector, which is predetermined, to each of the occupants.

According to yet another aspect of the present application, an occupant position estimating method executed by at least one processor to estimate a position of each of occupants in a vehicle cabin of a vehicle is provided. The occupant position estimating method includes controlling each of radars that transmit and receive radio waves in the vehicle cabin to transmit radio waves, obtaining multiple received strengths of the radio waves that are received by the respective radars, obtaining a separated waveform for each of the occupants through a process to isolate components corresponding respectively to the occupants from waveforms each of which indicates a time variation of the multiple received strengths, identifying a number of the occupants in the vehicle cabin from the separated waveform for each of the occupants, identifying distances from each of the radars to the respective occupants in accordance with the identified number of the occupants based on a timing at which a human-specific fluctuation appears in the separated waveform for each of the occupants, estimating a position of each of the occupants in the vehicle cabin based on the distances from each of the radars to the respective occupants. The obtaining of the multiple received strengths includes separately obtaining reflection characteristic values that are received strengths of the radio waves that are transmitted by one of the radars and received by the one of the radars, and transmission characteristic values that are received strengths of the radio waves that are transmitted by one of the radars and received by another of the radars. The obtaining of the separated waveform includes obtaining the separated waveform through a process to isolate components corresponding respectively to the occupants from a waveform which indicates a time variation of the reflection characteristic values and a waveform which indicates a time variation of the transmission characteristic values.

According to yet another aspect of the present application, an occupant position estimating method executed by at least one processor to estimate a position of each of occupants in a vehicle cabin of a vehicle is provided. The occupant position estimating method includes controlling each of radars that transmit and receive radio waves in the vehicle cabin to transmit radio waves, obtaining multiple received strengths of the radio waves that are received by the respective radars, obtaining a separated waveform for each of the occupants through a process to isolate components corresponding respectively to the occupants from waveforms each of which indicates a time variation of the multiple received strengths, identifying a number of the occupants in the vehicle cabin from the separated waveform for each of the occupants, identifying distances from each of the radars to the respective occupants in accordance with the identified number of the occupants based on a timing at which a human-specific fluctuation appears in the separated waveform for each of the occupants, estimating a position of each of the occupants in the vehicle cabin based on the distances from each of the radars to the respective occupants. At least one of the radars is a mobile terminal that is carried by one of the occupants, and a position of the mobile terminal relative to the vehicle is identified by a system of the vehicle using a communication between the mobile terminal and at least three antennas disposed in the vehicle. The other of the radars is a radio wave detector that is disposed in the vehicle, and a position of the radio wave detector relative to the vehicle is predetermined. The estimating the position of each of the occupants includes estimating the position of each of the occupants in the vehicle cabin based on a distance from the position of the mobile terminal, which is identified by the system of the vehicle, to each of the occupants and a distance from the position of the at least one radio wave detector, which is predetermined, to each of the occupants.

### BRIEF DESCRIPTION OF THE DRAWINGS

FIG. 1 is a diagram showing an example of a schematic configuration of a vehicle system.

FIG. 2 is a diagram showing an example of a schematic configuration of a mobile terminal.

FIG. 3 is a diagram showing an example of a schematic configuration of a vehicle unit.

FIG. 4 is a diagram showing an example of the positions of UWB anchors.

FIG. 5 is a diagram for explaining an example of the waveform of an impulse signal.

FIG. 6 is a diagram showing an example of a time variation of a received signal strength of a reflective wave received by one of the UWB anchors.

FIG. 7 is a schematic diagram showing an example of paths where reflection characteristic values and transmission characteristic values are obtained.

FIG. 8 is a diagram showing an example of a waveform in which multiple received strength waveforms are superimposed.

FIG. 9 is a diagram explaining an example where specific fluctuations caused by multiple occupants are overlapped in the received strength waveforms.

FIG. 10 is a flowchart showing an example of an occupant position estimation process in a communication ECU.

FIG. 11 is a diagram showing an example of a schematic configuration of a vehicle system.

FIG. 12 is a diagram showing an example of a schematic configuration of a mobile terminal.

FIG. 13 is a diagram showing an example of a schematic configuration of a vehicle unit.

FIG. 14 is a diagram showing an arrangement example of UWB anchors.

FIG. 15 is a diagram for explaining a scenario where radio waves are shielded by the seat in a vehicle positioned between an occupant and a mobile terminal.

FIG. 16 is a flowchart showing an example of a distance correction process in the communication ECU.

FIG. 17 is a flowchart showing a flow example of a terminal position re-estimation related process in the communication ECU.

### DETAILED DESCRIPTION OF EMBODIMENTS

To begin with, examples of relevant techniques will be described.

All occupants in a vehicle cabin are required to wear seat belts in many countries. Accordingly, vehicle manufacturers are required to equip a system that warns the occupants who do not wear seat belts. To issue a warning for not wearing a seat belt, it is necessary to detect the seating position of each of the occupants. As a device for detecting the seating position, a technology using a pressure sensor embedded in the seating part of the seat is known. However, it takes costs to install dedicated pressure sensors in all seats to detect the seating position of each of the occupants.

There is another technology that attempts to detect the presence of an occupant in each seat in the vehicle cabin without using a dedicated camera and sensor. In the technology, the presence of an occupant is detected using transmission and reception of Ultra-Wide Band (UWB) wireless radio signals between two terminals placed in the vehicle cabin. Specifically, the presence of the occupant is detected by determining whether the occupant is on board based on variation in received power value and delay spread value of the radio signals. In the technology, the presence of the occupant in each seat of the vehicle is detected based on the difference in the combination of the arrangement positions of the two terminals.

The variations in the received power value and delay spread value of the radio signals, which are used in the above technology, may not differ significantly between the human body and luggage. Thus, the technology may mistakenly detect large luggage within the detection range in the vehicle as a human. Additionally, it is not envisioned to detect and distinguish multiple occupants.

It is an objective of the present disclosure to provide an occupant position estimating device and an occupant position estimating method for estimating the position of each of the occupants in the vehicle more accurately while distinguishing between the occupants.

The above objective is achieved through the combination of features described in the independent claims, while the dependent claims define further advantageous specific examples of the disclosure.

According to one aspect of the present disclosure, an occupant position estimating device to estimate a position of each of occupants in a vehicle cabin of a vehicle is provided. The occupant position estimating device includes a transmission control unit, a received strength obtaining unit, a separating unit, a headcount identifying unit, a distance identifying unit, and an occupant position estimating unit. The transmission control unit controls each of radars to transmit radio waves. The radars transmit and receive radio waves in the vehicle cabin. The received strength obtaining unit obtains multiple received strengths of the radio waves that are received by the respective radars. The separating unit obtains a separated waveform for each of the occupants through a process to isolate components corresponding respectively to the occupants from waveforms each of which indicates a time variation of the multiple received strengths that are received by the received strength obtaining unit. The headcount identifying unit identifies a number of the occupants in the vehicle cabin from the separated waveform of each of the occupants. The distance identifying unit identifies distances from each of the radars to the respective occupants in accordance with the number of the occupants identified by the headcount identifying unit based on a timing at which a human-specific fluctuation appears in the separated waveform. The occupant position estimating unit estimates a position of each of the occupants in the vehicle cabin based on the distances from each of the radars to the respective occupants. The received strength obtaining unit separately obtains reflection characteristic values that are received strengths of the radio waves that are transmitted by one of the radars and received by the one of the radars, and transmission characteristic values that are received strengths of the radio waves that are transmitted by one of the radars and received by another of the radars. The separating unit is configured to obtain the separated waveform for each of the occupants through a process to isolate the components corresponding respectively to the occupants from a waveform which indicates a time variation of the reflection characteristic values and a waveform which indicates a time variation of the transmission characteristic values. The headcount identifying unit identifies the number of the occupants in the vehicle cabin based on the separated waveform of each of the occupants.

According to another aspect of the present disclosure, an occupant position estimating device to estimate a position of each of occupants in a vehicle cabin of a vehicle is provided. The occupant position estimating device includes a transmission control unit, a received strength obtaining unit, a separating unit, a headcount identifying unit, a distance identifying unit, and an occupant position estimating unit. The transmission control unit controls each of radars to transmit radio waves. The radars transmit and receive radio waves in the vehicle cabin. The received strength obtaining unit obtains multiple received strengths of the radio waves that are received by the respective radars. The separating unit obtains a separated waveform for each of the occupants through a process to isolate components corresponding respectively to the occupants from waveforms each of which indicates a time variation of the multiple received strengths that are received by the received strength obtaining unit. The headcount identifying unit identifies a number of the occupants in the vehicle cabin from the separated waveform of each of the occupants. The distance identifying unit identifies distances from each of the radars to the respective occupants in accordance with the number of the occupants identified by the headcount identifying unit based on a timing at which a human-specific fluctuation appears in the separated waveform. The occupant position estimating unit estimates a position of each of the occupants in the vehicle cabin based on the distances from each of the radars to the respective occupants. At least one of the radars is a mobile terminal that is carried by one of the occupants. A position of the mobile terminal relative to the vehicle is identified by a system of the vehicle using a communication between the mobile terminal and at least three antennas disposed in the vehicle. The other of the radars is a radio wave detector that is disposed in the vehicle. A position of the radio wave detector relative to the vehicle is predetermined. The occupant position estimating unit is configured to estimate the position of each of the occupants in the vehicle cabin based on a distance from the position of the mobile terminal, which is identified by the system of the vehicle, to each of the occupants and a distance from the position of the at least one radio wave detector, which is predetermined, to each of the occupants.

According to yet another aspect of the present application, an occupant position estimating method executed by at least one processor to estimate a position of each of occupants in a vehicle cabin of a vehicle is provided. The occupant position estimating method includes controlling each of radars that transmit and receive radio waves in the vehicle cabin to transmit radio waves, obtaining multiple received strengths of the radio waves that are received by the respective radars, obtaining a separated waveform for each of the occupants through a process to isolate components corresponding respectively to the occupants from waveforms each of which indicates a time variation of the multiple received strengths, identifying a number of the occupants in the vehicle cabin from the separated waveform for each of the occupants, identifying distances from each of the radars to the respective occupants in accordance with the identified number of the occupants based on a timing at which a human-specific fluctuation appears in the separated waveform for each of the occupants, estimating a position of each of the occupants in the vehicle cabin based on the distances from each of the radars to the respective occupants. The obtaining of the multiple received strengths includes separately obtaining reflection characteristic values that are received strengths of the radio waves that are transmitted by one of the radars and received by the one of the radars, and transmission characteristic values that are received strengths of the radio waves that are transmitted by one of the radars and received by another of the radars. The obtaining of the separated waveform includes obtaining the separated waveform for each of the occupants through a process to isolate components corresponding respectively to the occupants from a waveform which indicates a time variation of the reflection characteristic values and a waveform which indicates a time variation of the transmission characteristic values.

According to yet another aspect of the present application, an occupant position estimating method executed by at least one processor to estimate a position of each of occupants in a vehicle cabin of a vehicle is provided. The occupant position estimating method includes controlling each of radars that transmit and receive radio waves in the vehicle cabin to transmit radio waves, obtaining multiple received strengths of the radio waves that are received by the respective radars, obtaining a separated waveform for each of the occupants through a process to isolate components corresponding respectively to the occupants from waveforms each of which indicates a time variation of the multiple received strengths, identifying a number of the occupants in the vehicle cabin from the separated waveform for each of the occupants, identifying distances from each of the radars to the respective occupants in accordance with the identified number of the occupants based on a timing at which a human-specific fluctuation appears in the separated waveform for each of the occupants, estimating a position of each of the occupants in the vehicle cabin based on the distances from each of the radars to the respective occupants. At least one of the radars is a mobile terminal that is carried by one of the occupants, and a position of the mobile terminal relative to the vehicle is identified by a system of the vehicle using a communication between the mobile terminal and at least three antennas disposed in the vehicle. The other of the radars is a radio wave detector that is disposed in the vehicle, and a position of the radio wave detector relative to the vehicle is predetermined. The estimating of the position of each of the occupants includes estimating the position of each of the occupants in the vehicle cabin based on a distance from the position of the mobile terminal, which is identified by the system of the vehicle, to each of the occupants and a distance from the position of the at least one radio wave detector, which is predetermined, to each of the occupants.

According to the above configurations, the position of each of the occupants in the vehicle cabin is estimated based on the distances from each of the radars to the respective occupants, thereby estimating the position of each of the occupants more accurately. The waveforms each of which indicates a time variation of the multiple received strengths of radio waves that are transmitted by each of the radars in the vehicle cabin and received by the respective radars express human-specific fluctuations at parts reflected by the occupants. The configuration of the present disclosure identifies the distances from each of the radars to the respective occupants based on timings at which the human-specific fluctuations appear in the waveforms. Thus, it is possible to avoid mistakenly estimating the position of the luggage as the position of the occupant. From this point as well, the position of each of the occupants in the vehicle cabin is estimated more accurately. In addition, the number of occupants in the vehicle cabin is identified based on the separated waveforms which are obtained through a process to isolate components for the occupants contained in the waveforms each indicating a time variation of multiple received strengths for each of the radars. Thus, the position of each of the occupants in the vehicle cabin can be estimated based on the separated waveforms for the occupants. As a result, the position of each of the occupants can be estimated more accurately while distinguishing between the occupants.

Multiple embodiments for the disclosure will be described with reference to the drawings. For the sake of convenience in explanation, parts that have the same functions as those shown in the figures used in previous descriptions will be denoted by the same reference numerals among the multiple embodiments, and their descriptions may be omitted. For parts with the same reference numerals, the explanations in other embodiments can be referred to.

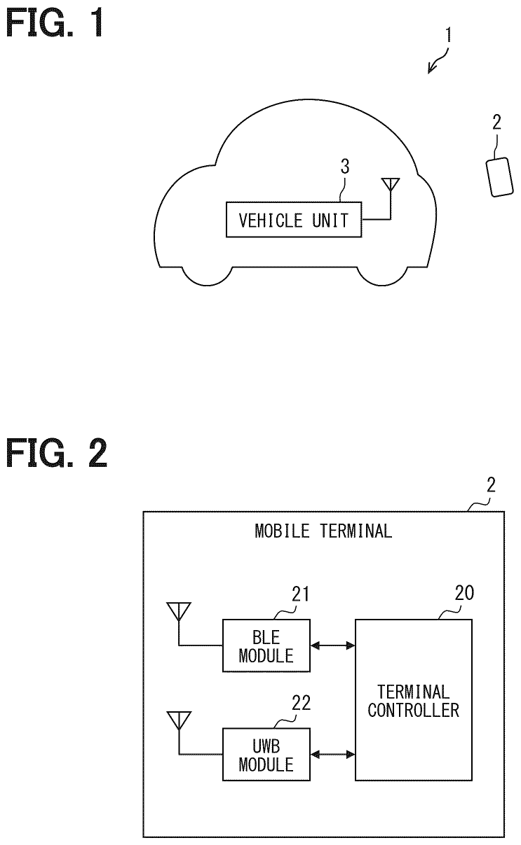

(First embodiment)<Schematic Configuration of Vehicle System 1> Hereinafter, a present embodiment will be described with reference to the drawings. As shown in FIG. 1, a vehicle system 1 includes a mobile terminal 2 and a vehicle unit 3.

The mobile terminal 2 is an information processing terminal such as a multifunctional mobile phone. A multifunctional mobile phone can also be referred to as a smartphone. The mobile terminal 2 is carried by a user. Hereinafter, the mobile terminal 2 is a multifunctional mobile phone as an example.

The vehicle unit 3 is used in a vehicle. Details of the vehicle unit 3 will be described later. The vehicle using the vehicle unit 3 may be an automobile. The vehicle using the vehicle unit 3 is hereinafter referred to as a subject vehicle. The mobile terminal 2 is carried by an occupant in the subject vehicle. It should be noted that the state “carried by the occupant” is not strictly limited to the state of being carried by the occupant. The state “the mobile terminal 2 is carried by the occupant” includes situations where the occupant is not actually carrying the mobile terminal 2, such as when the mobile terminal 2 is placed in a location different from the seating position of the occupant in the subject vehicle and when the mobile terminal 2 is left behind in the subject vehicle.

<Schematic Configuration of Mobile Terminal 2> The following will describe the configuration of the mobile terminal 2 with reference to FIG. 2. As shown in FIG. 2, the mobile terminal 2 includes a terminal controller 20, a BLE module 21, and a UWB module 22. In this embodiment, for convenience, descriptions of configurations that are not related to the present disclosure are omitted.

The BLE module 21 is a communication module capable of performing short-range wireless communication conforming to Bluetooth Low Energy (Bluetooth is a registered trademark). Bluetooth Low Energy is abbreviated as BLE. The BLE module 21 may be configured, for example, with an IC, an antenna, a communication circuit, and the like. The BLE module 21 establishes a communication connection with the vehicle unit 3 and performs short-range wireless communication with the vehicle unit 3.

The BLE module 21 periodically scans and receives advertising packets transmitted periodically from the vehicle unit 3. The BLE module 21 that has received the advertising packets transmits a connection request to the vehicle unit 3. When this connection request is accepted, communication connection between the BLE module 21 and the vehicle unit 3 is established.

The UWB module 22 is a communication module capable of performing short-range wireless communication using the Ultra Wide Band—Impulse Radio (UWB-IR) method. Hereafter, UWB-IR short-range wireless communication is referred to as UWB communication. The UWB communication can also be described as ultra-wideband wireless communication. The UWB module 22 may be configured, for example, with an IC, an antenna, a communication circuit, and the like.

The UWB module 22 performs the UWB communication by transmitting and receiving impulse-like radio waves (hereinafter referred to as “impulse signals”). An impulse signal used in the UWB communication is a signal having an extremely short pulse width. For example, the impulse signal has a pulse width of 2 ns. The impulse signals used in the UWB communication are signals with a bandwidth of 500 MHz or higher (i.e., ultra-wide bandwidth). Frequency bands that can be used for the UWB communication (hereinafter, referred to as the UWB band) include 3.1 GHz to 10.6 GHz, 3.4 GHz to 4.8 GHz, 7.25 GHz to 10.6 GHz, 22 GHz to 29 GHz, and the like. The UWB module 22 receives an impulse signal transmitted by the vehicle unit 3 and transmits back a response signal corresponding to the impulse signal.

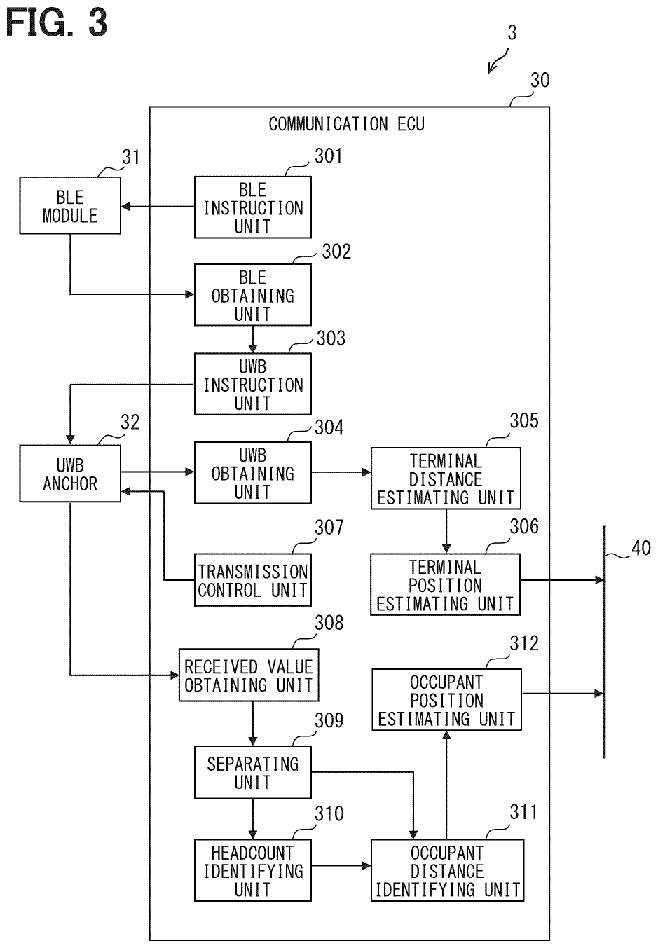

The terminal controller 20 includes, for example, a processor, a memory, I/O, and a bus connecting between them. The terminal controller 20 executes various processes by executing control programs stored in the memory. The terminal controller 20 executes various processes related to the control of radio wave transmission and reception by the BLE module 21 and the UWB module 22. The memory is a non-transitory tangible storage medium which non-temporary stores computer-readable programs and data. Further, the non-transitory tangible storage medium is implemented as a semiconductor memory or the like.

<Schematic Configuration of Vehicle Unit 3> The following will describe an example of a schematic configuration of the vehicle unit 3 with reference to FIG. 3. As shown in FIG. 3, the vehicle unit 3 includes a communication ECU 30, a BLE module 31, and a UWB anchor 32. For example, the communication ECU 30 may be connected to an in-vehicle LAN 40. In addition, the BLE module 31 and the UWB anchor 32 may be connected to the communication ECU 30.

The BLE module 31 is a communication module including, for example, an IC, an antenna, a communication circuit, and the like. The BLE module 31 performs short-range wireless communication conforming to BLE according to instructions by the communication ECU 30.

The UWB anchor 32 is a communication module including, for example, an IC, an antenna, a communication circuit, and the like. The UWB anchor 32 performs short-range wireless communication using the UWB-IR method according to instructions by the communication ECU 30. In other words, the UWB anchor 32 performs UWB communication. The UWB anchor 32 performs UWB communication with the mobile terminal 2. The UWB anchor 32 includes multiple UWB anchors 32 provided at multiple locations inside and outside a vehicle cabin of the subject vehicle. For example, the UWB anchors 32 outside the vehicle cabin may be arranged respectively near the left and right corners of the front and rear ends of the subject vehicle. The UWB anchors 32 in the vehicle cabin may be three. Hereinafter, an example where the three UWB anchors 32 are placed inside the vehicle cabin will be explained.

The UWB anchors 32 in the vehicle cabin have functions as radars. The functions as radars are to transmit radio waves (i.e., transmission waves) and receive reflected waves reflected from objects in the vehicle cabin. The UWB anchors 32, for example, can achieve the functions as radars by using an IC that can quickly switch between transmission and reception of radio waves. In the following, the UWB anchors 32 in the vehicle cabin will be referred to as the UWB anchors 320. Each of the UWB anchors 320 corresponds to a radar or a radio wave detector. The UWB anchors 320 may transmit impulse signals used in UWB communication even when used as radars.

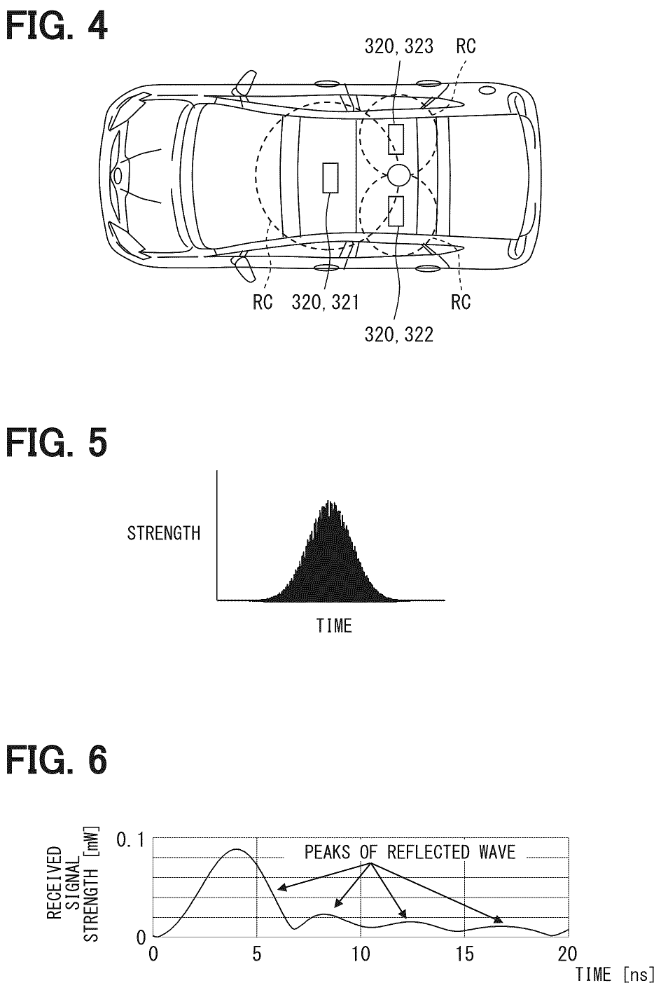

Here, an example of the arrangement of the UWB anchors 320 in this embodiment will be described with reference to FIG. 4. FIG. 4 is a diagram showing an example of the arrangement of the UWB anchors 320 (321 to 323). In the following, the three UWB anchors 320 are represented as UWB anchors 321, 322, and 323, respectively. The UWB anchor 321 is placed in the front center area of the vehicle cabin. The UWB anchor 322 is placed in the left rear area of the vehicle cabin. The UWB anchor 323 is placed in the right rear area of the vehicle cabin. The UWB anchors 320 may be provided on the ceiling of the vehicle cabin.

Each of the UWB anchors 32 is equipped with a timer that measures the elapsed time (hereinafter referred to as a round-trip time) from the transmission of the impulse signal to the reception of the impulse signal as a response signal to this impulse signal. The UWB anchor 32 measures the round-trip time using the timer. The UWB anchor 32 outputs the measured round-trip time to the communication ECU 30. When the UWB anchor 320 is used as a radar, the UWB anchor 320 transmits an impulse signal and then receives the reflected wave of the impulse signal. Additionally, when the UWB anchor 320 is used as a radar, the UWB anchor 320 receives impulse signals transmitted by other UWB anchors 320. The UWB anchors 320 output received strength of the reflected waves to the communication ECU 30. The received strength of the reflected waves may be rephrased as Received Signal Strength Indication (RSSI).

The communication ECU 30 includes, for example, a processor, a memory, I/O, and a bus connecting between them. The communication ECU 30 executes various processes by executing control programs stored in the memory. The communication ECU 30 executes various processes related to the control of radio wave transmission and reception by the BLE module 31 and the UWB anchor 32. The communication ECU 30 executes a process to estimate the position of the mobile terminal 2 relative to the vehicle. The communication ECU 30 executes a process to estimate the positions of the occupants in the vehicle cabin. The memory is a non-transitory tangible storage medium that non-temporary stores computer-readable programs and data. The non-transitory tangible storage medium may be provided by a semiconductor memory or a magnetic disk, for example. The communication ECU 30 corresponds to an occupant position estimating device. The details of the communication ECU 30 will be described below.

<Schematic Configuration of Communication ECU 30> The following will describe an example of the schematic configuration of the communication ECU 30 with reference to FIG. 3. As shown in FIG. 3, the communication ECU 30 includes, as functional blocks, a BLE instruction unit 301, a BLE obtaining unit 302, a UWB instruction unit 303, a UWB obtaining unit 304, a terminal distance estimating unit 305, a terminal position estimating unit 306, a transmission control unit 307, a received value obtaining unit 308, a separating unit 309, a headcount identifying unit 310, an occupant distance identifying unit 311, and an occupant position estimating unit 312. Execution of a process of each functional block of the communication ECU 30 by the computer corresponds to execution of an occupant position estimating method. In addition, a part or all of the functions executed by the communication ECU 30 may be configured as a hardware, such as one or more of ICs or the like. Alternatively, a part or all of functional blocks of the communication ECU 30 may be implemented by a combination of a software executed by at least one processor and a hardware.

The BLE instruction unit 301 causes the BLE module 31 to transmit advertising packets. For example, the BLE instruction unit 301 may cause the BLE module 31 to periodically transmit advertising packets after a certain period has elapsed since the vehicle was parked and all doors of the vehicle have been locked. The periodic transmission of the advertising packets may be end at a timing when the vehicle starts moving.

The BLE obtaining unit 302 obtains information related to wireless communication with the mobile terminal 2, which is received by the BLE module 31. The BLE obtaining unit 302 obtains information received by the BLE module 31 through short-range wireless communication with the BLE module 21. This process may be executed when a connection is established between the BLE module 31 and the BLE module 21 of the mobile terminal 2 that has received the advertising packets.

The UWB instruction unit 303 causes the UWB anchors 32 to transmit impulse signals. As an example, the UWB instruction unit 303 causes the UWB anchors 32 provided in the subject vehicle to sequentially transmit impulse signals one by one. The UWB instruction unit 303 may cause the UWB anchors 32 to start transmitting impulse signals when the connection described above is or has been established. According to this, the UWB anchors 32 can avoid needlessly transmitting impulse signals when the mobile terminal 2 does not exist around the subject vehicle. The UWB instruction unit 303 may determine that the above-mentioned connection is or has been established, for example, from the information obtained by the BLE obtaining unit 302. The establishment of the connection may be determined from the fact that the BLE module 31 receives the connection request sent by the BLE module 21. The establishment of the connection may be determined from the fact that the BLE module 31 receives the information transmitted through the short-range wireless communication that is performed after the connection establishment.

The UWB obtaining unit 304 obtains information related to UWB communication, which is received by the UWB anchors 32. The UWB communication is performed between the UWB module 22 of the mobile terminal 2 and the UWB anchors 32. The UWB obtaining unit 304 obtains the round-trip time output from each of the UWB anchors 32 provided in the vehicle.

The terminal distance estimating unit 305 estimates the distance from each of the UWB anchors 32 to the mobile terminal 2 based on the impulse signals that are transmitted and received between the mobile terminal 2 and the UWB anchors 32. The distance from each of the UWB anchors 32 to the mobile terminal 2 is referred to as a terminal distance. The terminal distance estimating unit 305 estimates the terminal distance from the UWB anchors 32 that have received the response signals among the multiple UWB anchors 32. The terminal distance estimating unit 305 may estimate the terminal distance using the round-trip time obtained by the UWB obtaining unit 304. More specifically, the terminal distance estimating unit 305 calculates the propagation time by subtracting the internal processing time at the mobile terminal 2 in UWB communication from the round-trip time, and then dividing the resulting value by 2. Then, the terminal distance estimating unit 305 multiplying the calculated propagation time by the speed of light to get a value as the estimated terminal distance. The standard value of the internal processing time may be stored in the non-volatile memory of the communication ECU 30 in advance.

The terminal position estimating unit 306 estimates the position of the mobile terminal 2 relative to the vehicle using the terminal distance estimated by the terminal distance estimating unit 305. The position of the mobile terminal 2 relative to the vehicle is hereinafter referred to as a terminal position. The terminal position estimating unit 306 may estimate the terminal position using the terminal distances from the three UWB anchors 32. As for the selection of the three UWB anchors 32, the three with the shortest terminal distances should be selected. One example of estimating the terminal position is as follows.

First, in a coordinate system of a horizontal plane that takes the reference point of the vehicle as the origin (hereinafter referred to as the plane coordinate system), three circles having centers at the positions of the three UWB anchors 32 and radii equivalent to the respective terminal distances are drawn. Then, based on these three circles, the terminal position is estimated by trilateration. Trilateration can also be referred to as triangulation. The reference point of the vehicle can be determined as appropriate. For example, the reference point may be a center of the rear axle in the vehicle width direction. The positions of the UWB anchors 32 relative to the vehicle may be stored in the non-volatile memory of the communication ECU 30 in advance. The terminal position estimating unit 306 outputs the estimated terminal position to the in-vehicle LAN 40. This terminal position is used, for example, in digital key systems and notifications of forgetting the mobile terminal 2 inside the vehicle.

The transmission control unit 307 causes three of the UWB anchors 320 as radars to transmit radio waves (transmission waves). In the example of this embodiment, impulse signals used in UWB communication are transmitted. The transmission control unit 307 switches the UWB anchors 320 to operate in a predetermined cycle to avoid interference between the UWB anchors 320.

The transmission control unit 307 causes each of the UWB anchors 320 to transmit transmission waves during a uniquely given transmission period that is set for the respective UWB anchors 320. The transmission control unit 307 causes each of the UWB anchors 320 to transmit radio waves multiple times, with each transmission time being shorter than the transmission period. The radio waves have different transmission signal strength (see FIG. 5). FIG. 5 is a diagram showing an example of a pulse waveform of an impulse signal transmitted by each of the UWB anchors 320. As shown in FIG. 5, the envelope formed by the transmission signal strength of the impulse signal has a mountain-like shape. In other words, the transmission signal strength is lowest at the beginning and end of the transmission time, and highest in the center of the transmission time. The transmission control unit 307 causes the impulse signals shown in FIG. 5 to be transmitted multiple times at predetermined intervals during the transmission period. The predetermined interval at this time is set to a value sufficiently small relative to the human breathing and body movement cycle. The predetermined interval may be set, for example, to 100 ms. The following explanation will use the example where the predetermined interval is 100 ms. The processes executed by the transmission control unit 307 corresponds to a transmission control process.

The received value obtaining unit 308 obtains received strength of the transmission waves received by each of the UWB anchors 320. In the example of this embodiment, the received value obtaining unit 308 obtains received values received by the UWB anchors 321, 322, and 323. The received values are at least the received strength of the received radio waves (i.e., reflected waves). The received values may include, for example, radio wave arrival times, the frequency of radio waves, and signals contained in radio waves. The received value obtaining unit 308 corresponds to a received strength obtaining unit. Also, the process executed by the received value obtaining unit 308 corresponds to a received strength obtaining process. The radio wave arrival time is the time elapsed from the transmission of the impulse signal by the UWB anchor 320 to the reception of the reflected signal. For example, regarding the radio wave received by the UWB anchor 321 during the transmission period of the UWB anchor 321, the transmission of the impulse signal by the UWB anchor 321 serves as the reference for the radio wave arrival time. Regarding the radio wave received by the UWB anchor 322 during the transmission period of the UWB anchor 321, the transmission of the impulse signal by the UWB anchor 321 serves as the reference for the radio wave arrival time. Regarding the radio wave received by the UWB anchor 322 during the transmission period of the UWB anchor 322, the transmission of the impulse signal by the UWB anchor 322 serves as the reference for the radio wave arrival time.

Here, an example of a time variation of the radio wave strength received by the UWB anchor 320 will be described with reference to FIG. 6. FIG. 6 shows a waveform of strength of the reflected wave received within 20 ns from the transmission of the impulse signal. As shown in FIG. 6, the waveform obtained is a superimposed waveform of signals reflected from various objects. The waveform in the example shown in FIG. 6 has four peaks, indicating objects in four locations.

The received value obtaining unit 308 may obtain, as the received strength, reflection characteristic values (reflection characteristic waveforms) and the transmission characteristic values (transmission characteristic waveforms) separately. According to this, even if the number of the UWB anchors 320 is less than the capacity of the vehicle, the separating unit 309, which will be described later, can accurately isolate the components for the occupants. The reflection characteristic value is the received strength of the radio wave that is transmitted by the UWB anchor 320 and received by the own UWB anchor 320. The transmission characteristic value is the received signal strength of the radio wave that is transmitted by the UWB anchor 320 and received by other UWB anchor 320. The received value obtaining unit 308 may distinguish between the reflection characteristic value from the transmission characteristic value based on which UWB anchor 320 transmits the radio wave at a certain timing and which UWB anchor 320 receives the reflected wave.

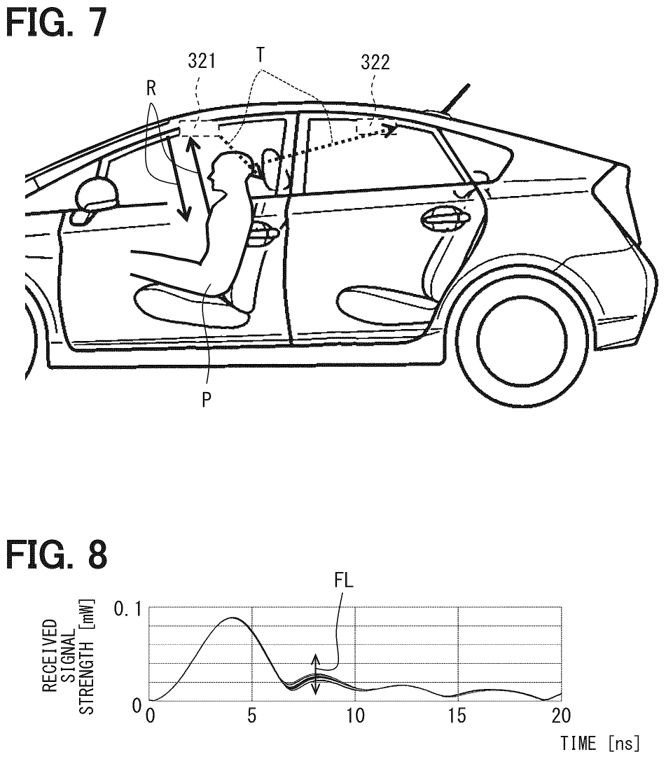

Here, the reflection characteristic value and transmission characteristic value will be explained using FIG. 7. FIG. 7 is a schematic diagram showing an example of paths in which the reflection characteristic value and the transmission characteristic value are obtained. FIG. 7 shows the scenario where the radio waves transmitted by the UWB anchor 321 are received by the UWB anchor 321 and the UWB anchor 322 as an example. The solid lines indicated by R in FIG. 7 show the path of the radio wave transmitted by the UWB anchor 321 and received by the UWB anchor 321. The dotted line indicated by T in FIG. 7 shows the path of the radio wave transmitted by the UWB anchor 321 and received by the UWB anchor 322. The reference sign P in FIG. 7 indicates the occupant. The reflection characteristic value is the received strength of the reflected wave, which is the radio wave transmitted by the UWB anchor 321, reflected by the occupant P, and received by the same UWB anchor 321. The transmission characteristic value is the received strength of the reflected wave, which is the radio wave transmitted by the UWB anchor 321, reflected by the occupant P, and received by another UWB anchor 322.

The received value obtaining unit 308 obtains a number of reflection characteristic values and transmission characteristic values equal to the seating capacity of the vehicle. In the example of this embodiment, the seating capacity in the vehicle is 5. The received value obtaining unit 308 obtains, for example, three reflection characteristic values and two transmission characteristic values. The first reflection characteristic value is the received strength of the radio wave that is transmitted by the UWB anchor 321 and received by the UWB anchor 321. The second reflection characteristic value is the received strength of the radio wave that is transmitted by the UWB anchor 322 and received by the UWB anchor 322. The third reflection characteristic value is the received strength of the radio wave that is transmitted by the UWB anchor 323 and received by the UWB anchor 323. The first transmission characteristic value is the received strength of the radio wave that is transmitted by the UWB anchor 321 and received by the UWB anchor 322. The second transmission characteristic value is the received strength of the radio wave transmitted by the UWB anchor 321 and received by the UWB anchor 323.

Here, the method for estimating the position of an occupant as a prerequisite will be described. FIG. 6 shows a waveform (hereinafter, referred to as a received strength waveform) indicating a time variation of the received signal strength obtained by the received value obtaining unit 308. FIG. 8 shows a composite waveform which is a composite of received strength waveforms obtained from multiple transmission of impulse signals at intervals that are sufficiently small with respect to human breathing and body movement cycles. Humans exhibit breathing and body movements while inanimate objects such as luggage does not exhibit. The composite waveform of the multiple received strength waveforms shows a human-specific fluctuation such as breathing and body movements. The fluctuation shown by FL in FIG. 8 indicates a human-specific fluctuation (hereinafter simply referred to as specific fluctuation). The timing at which this specific fluctuation starts can be regarded as the timing at which the impulse signal is reflected by the human body. Thus, the distance from the UWB anchor 320 to the occupant can be identified based on the timing. The distance from the UWB anchor 320 to the occupant is hereinafter referred to as an occupant distance. The occupant distance is identified for two or three UWB anchors 320, and the occupant position is estimated using the occupant distances by two-point or three-point positioning. The reference signs RC in FIG. 4 show ranging circles based on the occupant distances specified for each of the UWB anchors 320. If the occupant position is identified with two-point positioning, the two points are estimated as candidates for the occupant position. Therefore, when using two-point positioning, the two UWB anchors 320 may be arranged so that one of the candidates is always outside the vehicle. This allows the occupant position in the vehicle cabin to be estimated to a single point even with two-point positioning. It should be noted that estimating the occupant position using three-point positioning is easier and more accurate.

According to the above configuration, the occupant position can be identified more accurately. In addition, according to the above configuration, the distance from each of the UWB anchors 320 to the occupant is specified based on the timing at which the specific fluctuation appears. Thus, it is possible to avoid mistakenly estimating the position of luggage as the position of the occupant. From this point, the occupant position in the vehicle cabin can be estimated more accurately.

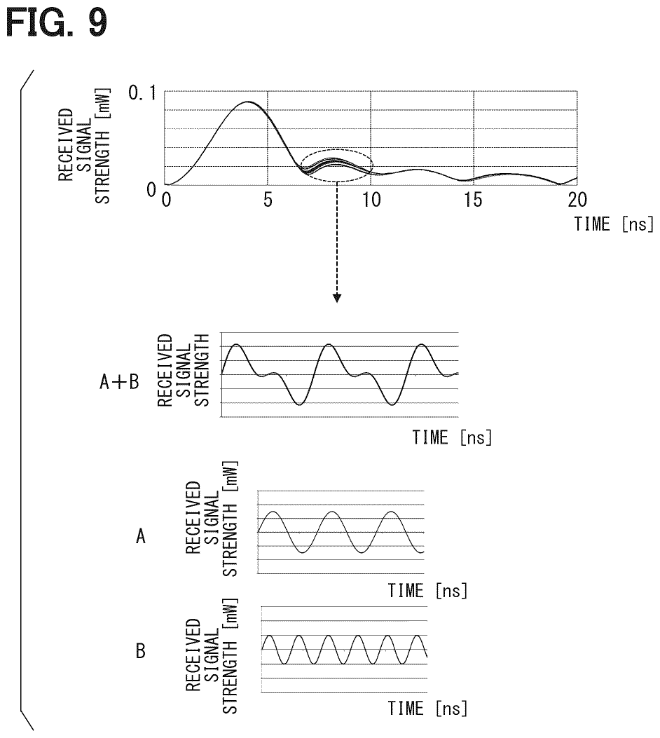

When the number of occupants is one, the position of the occupant is estimated by the above-mentioned estimating method. However, the number of occupants is not always one. It is conceivable to identify the number of occupants from the number of areas where specific fluctuations appear in the received strength waveforms. However, it is sometimes difficult to determine the number of occupants from the number of areas where specific fluctuations appear in the received signal waveforms. For example, this could occur when the distances from the UWB anchor 320 to the occupants are equivalent. This could also occur when specific fluctuations for the occupants overlap due to the influence of multipath. In such cases, as shown in FIG. 9, the specific fluctuation that appear to be for one person includes the specific fluctuations for multiple people. The middle graph of the FIG. 9 labeled A+B shows a waveform in which the specific fluctuations of multiple people are superimposed. The lower graph of FIG. 9 labeled A and the lower graph labeled B show the waveforms of specific fluctuations for different occupants, respectively. To address such issues and make it easier to identify the number of occupants, the separating unit 309 performs following processing.

The separating unit 309 obtains separated waveforms for the occupants through a process to isolate components corresponding respectively to the occupants from a waveform each of which indicates a time variation of multiple received strength of radio waves received by each of the UWB anchors 320. The processing executed by the separating unit 309 corresponds to a separating process. The multiple received strength of radio waves received by each of the UWM anchors 320 are obtained by the received value obtaining unit 308. As for the received strength, both reflection characteristic values and transmission characteristic values may be used. As a process for isolating the components for the occupants from the waveform (i.e., the composite waveform formed by superimposing the received strength waveforms), independent component analysis may be used. It is also possible to use other means than independent component analysis to isolate the components for the occupants from the waveform. In the following, isolating the components for the occupants from the waveform with the independent component analysis will be explained as an example.

When using independent component analysis, input waveforms are the waveforms each indicating a variation of the multiple received strength received by the received value obtaining unit 308. Through independent component analysis, the input waveforms are separated into a number of output waveforms equal to the number of input waveforms. Generally, the number of input waveforms is equivalent to the number of output waveforms. For example, the seating capacity of the vehicle is 5 in this example and thus the maximum number of occupants in the vehicle cabin is 5. Therefore, five types of input waveforms are required to obtain the output waveforms for up to five occupants. In this embodiment, both the reflection characteristic values and the transmission characteristic values are used as the received strength. Thus, even when the number of the UWB anchors 320 is less than the capacity of the vehicle, the number of types of input waveforms equivalent to the capacity can be prepared. Therefore, the number of the occupants in the vehicle cabin can be identified while keeping the number of the UWB anchors 320 installed on the vehicle to a minimum. In cases where the seating capacity of the vehicle is three or fewer, it is also acceptable to use only the reflection characteristic values as the received strength.

In the received strength waveform for the UWB anchor 320, multipaths overlap as time progresses. That is, specific fluctuations for all the occupants in the vehicle cabin are superimposed in the later received strength waveform. Thus, the received value obtaining unit 308 may plot the received strength waveform every 100 ms, starting from the estimated time when the specific fluctuations for all the occupants are superimposed after the transmission of the impulse signal. As a result, the received value obtaining unit 308 obtains target received strength waveforms. Since the target received strength waveforms include specific fluctuations for the breathing of all the occupants in the vehicle cabin, the target received strength waveforms obtained from the UWB anchors 320 can be separated into the separated waveforms respectively for the occupants through independent component analysis. Hereinafter, the estimated time at which the waveforms with specific fluctuations for all the occupants are superimposed is referred to as a target time. The target time may be the time when the received signal waveform from the occupant of the seat farthest from the target UWB anchor 320 is obtained.

The headcount identifying unit 310 identifies the number of the occupants in the vehicle cabin based on the separated waveforms. The processing executed by the headcount identifying unit 310 corresponds to a headcount identifying process. The headcount identifying unit 310 groups the separated waveforms obtained by the separating unit 309 into a number equivalent to the seating capacity of the vehicle. The headcount identifying unit 310 may group the separated waveforms by setting a threshold for the correlation coefficient and classifying the waveforms with the correlation coefficient above the threshold into the same group as belonging to the same person. Then, the number of the groups is identified as the number of the occupants in the vehicle cabin.

The occupant distance identifying unit 311 identifies the occupant distances based on timings at which specific fluctuations appear in the separated waveforms. The occupant distance identifying unit 311 identifies the occupant distance for each of the occupants in accordance with the number of the occupants identified by the headcount identifying unit 310. The occupant distance identifying unit 311 corresponds to a distance identifying unit. In addition, the processing executed by the occupant distance identifying unit 311 corresponds to an occupant distance identifying process. The occupant distance identifying unit 311 may use fluctuations specific to body movements. However, it is preferable to use breathing-specific fluctuations because they are easier to distinguish from waveforms caused by luggage compared to fluctuations caused by body movement, thus improving robustness.

The occupant distance identifying unit 311 may identify the timings at which the specific fluctuations appear based on the waveforms before separated by the separating unit 309. The occupant distance identifying unit 311 may identify the timings at which the specific fluctuations appear based on the frequency characteristics corresponding to the specific fluctuations. The occupant distance identifying unit 311 identifies which of the separated waveforms obtained by the separating unit 309 configures a part of the waveform where the specific fluctuation appears. This identification may be performed with methods such as multiple regression analysis. Next, the occupant distance identifying unit 311 identifies timings at which specific fluctuations start for each of the separated waveforms grouped for different occupants. Then, the occupant distance identifying unit 311 may identify the occupant distances by Time of Flight (TOF) based on the time from transmitting the impulse signal to the identified timing. The occupant distance identified from the transmission characteristic value has less accuracy compared to the occupant distance identified from the reflection characteristic value. Therefore, it is preferable to use the reflection characteristic value to identify the occupant distance.

The occupant position estimating unit 312 estimates the position of each of the occupants based on the occupant distance from each of the UWB anchors 320 identified by the occupant distance identifying unit 311. The processing executed by the occupant position estimating unit 312 corresponds to the occupant position estimating process. Specifically, the occupant position estimating unit 312 estimates the position of each of the occupants by three-point positioning using the three occupant distances from the UWB anchors 321, 322, 323, similar to the estimation of the terminal position.

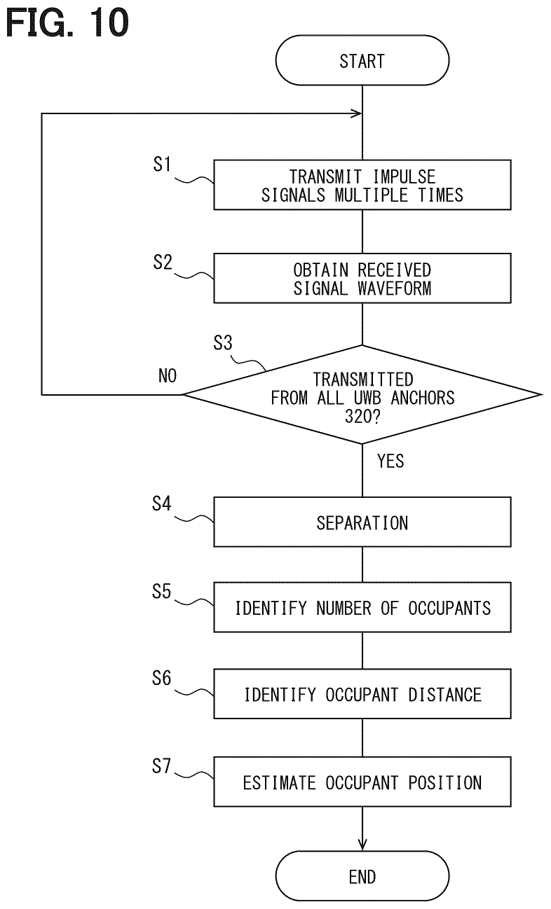

According to the above configuration, the position of each of the occupants in the vehicle cabin can be estimated using multiple waveforms for the occupants. As a result, the occupant position in the vehicle cabin can be estimated more accurately, and the occupant position for each of the occupants can be estimated while distinguishing between the occupants.

<Occupant Position Estimation Process by Communication ECU 30> Next, with reference to the flowchart in FIG. 10, an example of the procedure of the process related to occupant position estimation (hereinafter referred to as occupant position estimation process) in the communication ECU 30 will be described. The flowchart in FIG. 10, for example, may be executed repeatedly at regular intervals when the power switch of the vehicle is turned on. The power switch is a switch for starting the internal combustion engine or motor generator of the vehicle.

First, in step S1, the transmission control unit 307 controls the UMB anchor 320 to transmit multiple impulse signals at predetermined intervals. The transmission interval (the predetermined interval) is set to, for example, 100 ms. In S1, out of the three UWB anchors 320, only one UWB anchor 320 transmits impulse signals. In step S2, the received value obtaining unit 308 obtains the target received strength waveforms. Specifically, the received value obtaining unit 308 obtains the received strength waveforms that are plotted every 100 ms after the target time has elapsed since the impulse signal was transmitted from the UWB anchor 320.

In step S3, if the transmission of impulse signals from all the three UWB anchors 320 is completed (YES in S3), the process moves to step S4. On the other hand, if there are any UWB anchors 320 that have not finished transmitting the impulse signals (NO in S3), the process returns to S1 and repeats. In this case, the impulse signal is transmitted from the UWB anchor 320 in the next order in the pre-set transmission order. As an example, the transmission order may be in the order of the UWB anchor 321, the UWB anchor 322, and the UWB anchor 323.

In an example of the present embodiment, when the UWB anchor 321 transmits impulse signals, the target received strength waveforms received by the UWB anchors 321, 322, and 323 may be obtained in S2. When the UWB anchor 322 transmits impulse signals, it is sufficient to obtain the target received strength waveforms received by the UWB anchor 322 may be obtained in S2. When the UWB anchor 323 transmits impulse signals, it is sufficient to obtain the target received strength waveforms received by the UWB anchor 323 may be obtained in S2.

In step S4, the separating unit 309 separates the waveforms obtained in S3. For example, this separation may be performed by independent component analysis. In step S5, the headcount identifying unit 310 identifies the number of the occupants in the vehicle cabin based on the separated waveforms obtained in S4. In step S6, the occupant distance identifying unit 311 identifies the occupant distances based on the timings at which specific fluctuations appear in the separated waveforms obtained in S4. In S6, the occupant distances are specified respectively for the occupants in accordance with the number of the occupants identified in S5. In step S7, the occupant position estimating unit 312 estimates the position of each of the occupants in the vehicle cabin based on the occupant distances from each of the UWB anchors 320 identified in S6. Then, the occupant position estimation process ends.

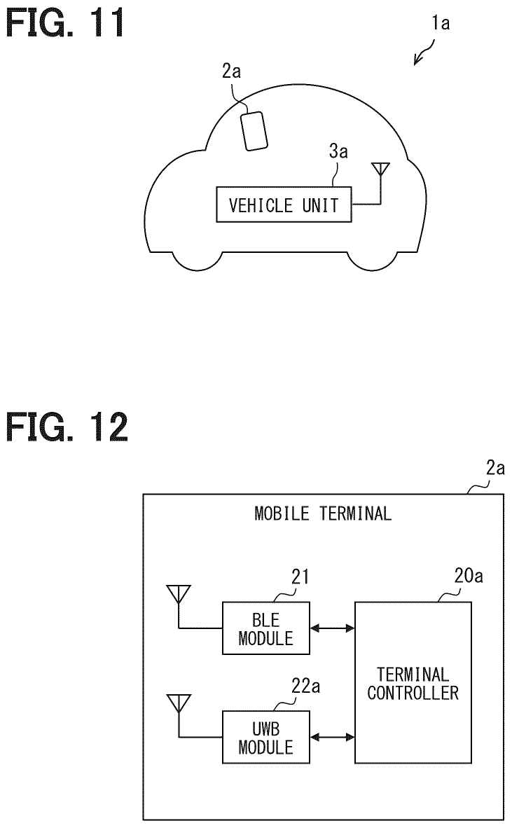

(Second Embodiment) The present disclosure is not limited to the configuration described in the first embodiment, and can also adopt the following configurations as a second embodiment. The followings will describe an example of a configuration of the second embodiment with reference to the accompanying drawings.

<Schematic Configuration of Vehicle System 1a> Hereinafter, a present embodiment will be described with reference to the drawings. As shown in FIG. 11, the vehicle system 1a includes a mobile terminal 2a and a vehicle unit 3a. The details of the mobile terminal 2a will be described later. The details of the vehicle unit 3a will be described later.

<Schematic Configuration of Mobile Terminal 2a> The following will describe the configuration of the mobile terminal 2a with reference to FIG. 12. As shown in FIG. 12, the mobile terminal 2a includes a terminal controller 20a, a BLE module 21, and a UWB module 22a. The mobile terminal 2a is different from the mobile terminal 2 of the first embodiment in including the terminal controller 20a instead of the terminal controller 20, and other portions of the mobile terminal 2a are similar to those of the mobile terminal 2 of the first embodiment.

The UWB module 22a differs from the UWB module 22 of the first embodiment in that the UWB module 22a essentially has the function as a radar described above, and other portions of the UWB module 22a are similar to those of the UWB module 22 of the first embodiment. The UWB module 22a can achieve the function as a radar, for example, by using an IC that can switch between the transmission and reception of radio waves quickly. Therefore, the mobile terminal 2a equipped with this UWB module 22a corresponds to the radar. The mobile terminal 2a may transmit impulse signals used in UWB communication even when using the mobile terminal 2a as a radar.

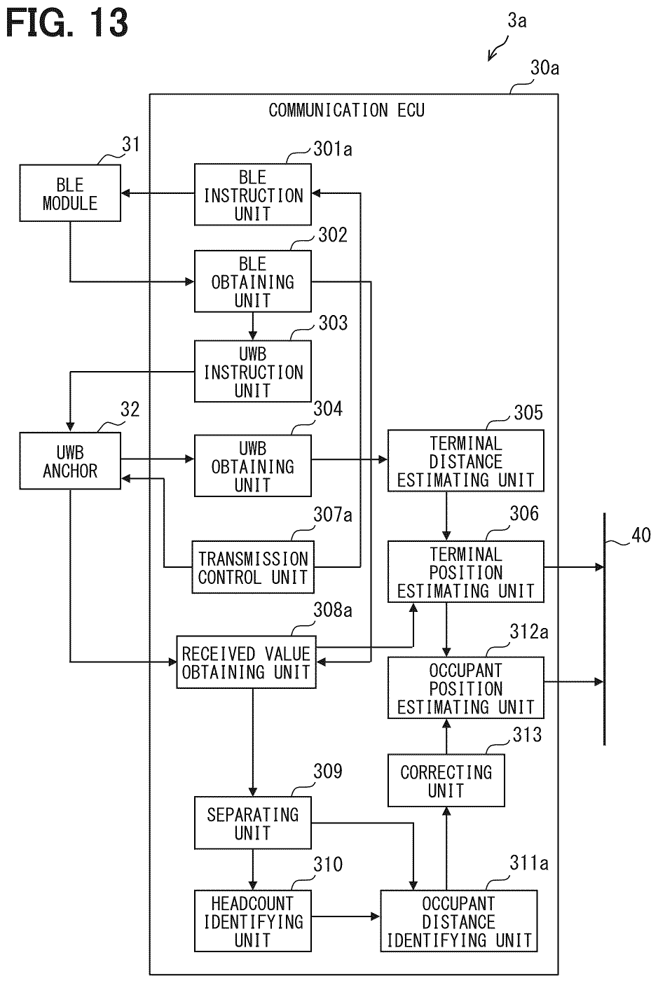

The terminal controller 20a differs from the terminal controller of the first embodiment in a part of the processes, and other portions of the terminal controller 20a are similar to those of the terminal controller of the first embodiment. The terminal controller 20a causes the UWB module 22a to serve as a radar according to instructions from the vehicle unit 3a. In other words, the terminal controller 20a causes the UWB module 22a to transmit radio waves as a radar. In this embodiment, the impulse signals used in UWB communication are transmitted. The terminal controller 20a may obtain instructions from the vehicle unit 3a via the BLE module 21. The terminal controller 20a obtains received strength of the reflected waves transmitted as a radar function. In this embodiment, the received value received by the UWB module 22a is obtained. The received value is at least the received signal strength of radio wave. The received value may include, for example, a radio wave arrival time, the frequency of the radio wave, and a signal contained in the radio wave. The terminal controller 20a sends the received value received by the UWB module 22a to the vehicle unit 3a. This transmission may be performed by the BLE module 21 or the UWB module 22a.

<Schematic Configuration of Vehicle Unit 3a> The following will describe an example of the schematic configuration of the vehicle unit 3a with reference to FIG. 13. As shown in FIG. 13, the vehicle unit 3a includes a communication ECU 30a, a BLE module 31, and a UWB anchor 32a. The vehicle unit 3a includes the communication ECU 30a instead of the communication ECU 30. The vehicle unit 3a includes UWB anchors 32a instead of the UWB anchors 32. The vehicle unit 3a is similar to the vehicle unit 3 of the first embodiment, except for these points.

The UWB anchors 32a differ from the UWB anchors 32 of the first embodiment in the arrangement in the subject vehicle. Other portions of the UWB anchors 32a are similar to those of the UWB anchors 32 of the first embodiment. Specifically, the UWB anchors 32a differ from the UWB anchors 320 of the first embodiment in the arrangement of the UWB anchors 320a, which are some of the UWB anchors 32a disposed in the vehicle cabin.

Here, an example of the arrangement of the UWB anchors 320a in this embodiment will be described with reference to FIG. 14. FIG. 14 is a diagram showing an example of the arrangement of the UWB anchors 320a (321, 324). In this embodiment, two UWB anchors 320a are provided in the vehicle. The two UWB anchors 320a are represented separately as the UWB anchor 321 and the UWB anchor 324. The UWB anchor 321 is placed at the front center of the vehicle cabin, similar to the first embodiment. The UWB anchor 324 is placed in the rear center of the vehicle cabin. The UWB anchors 320a may be provided on the ceiling inside the vehicle cabin. Each of the UWB anchors 320a corresponds to a radar or a radio wave detector.

The communication ECU 30a is similar to the communication ECU 30 of the first embodiment, except for some differences in processing. The communication ECU 30a corresponds to an occupant position estimating device. The details of the communication ECU 30a will be described below.

<Schematic Configuration of Communication ECU 30a> An example of the schematic configuration of the communication ECU 30a will be described with reference to FIG. 13. As shown in FIG. 13, the communication ECU 30a includes, as functional blocks, a BLE instruction unit 301a, a BLE obtaining unit 302, a UWB instruction unit 303, a UWB obtaining unit 304, a terminal distance estimating unit 305, a terminal position estimating unit 306, a transmission control unit 307a, a received value obtaining unit 308a, a separating unit 309, a headcount identifying unit 310, an occupant distance identifying unit 311a, an occupant position estimating unit 312a, and a correcting unit 313. The communication ECU 30a includes the BLE instruction unit 301a instead of the BLE instruction unit 301. The communication ECU 30a includes the transmission control unit 307a instead of the transmission control unit 307. The communication ECU 30a includes the received value obtaining unit 308a instead of the received value obtaining unit 308. The communication ECU 30a includes the occupant distance identifying unit 311a instead of the occupant distance identifying unit 311. The communication ECU 30a includes the occupant position estimating unit 312a instead of the occupant position estimating unit 312. The communication ECU 30a includes the correcting unit 313. The communication ECU 30a is similar to the communication ECU 30 of the first embodiment, except for these points. The processing of each functional block of the communication ECU 30a by the computer corresponds to the execution of an occupant position estimating method.

The BLE instruction unit 301a is similar to the BLE instruction unit 301 of the first embodiment, except for some differences in processing. The following describes the part of the processing of the BLE instruction unit 301a different from that of the BLE instruction unit 301. The BLE instruction unit 301a causes the UWB module 22a to transmit radio waves as a radar. The BLE instruction unit 301a performs this process according to the instructions of the transmission control unit 307a. The transmission control unit 307a causes two UWB anchors 320a and the mobile terminal 2a to transmit radio waves as a radar. In other words, the mobile terminal 2a is used as a radar. The mobile terminal 2a used as a radar may be, for example, the mobile terminal 2a registered as a key for the vehicle in a digital key system. The mobile terminal 2a is brought into the vehicle. Whether the mobile terminal 2a is brought into the vehicle or not can be determined by the terminal position estimated by the terminal position estimating unit 306 in the communication ECU 30a. In this embodiment, the impulse signals used in UWB communication are transmitted. The transmission control unit 307a is similar to the transmission control unit 307 of the first embodiment, except that the transmission control unit 307a controls the mobile terminal 2a to transmit radio waves as a radar. The transmission control unit 307 switches the source of radio waves in a predetermined cycle to prevent interference between the UWB anchors 320a and the mobile terminal 2a.

The received value obtaining unit 308a obtains the received strength of the radio waves received by the UWB anchors 320a and the mobile terminal 2a, respectively. In this embodiment, the received value obtaining unit 308a obtains the received values received by the UWB anchors 321 and 324. Additionally, the received value obtaining unit 308a obtains the received value received by the mobile terminal 2a. The received value obtaining unit 308a may obtain the received value received by the mobile terminal 2a via the BLE module 31 and the BLE obtaining unit 302. The received value obtaining unit 308a is similar to the received value obtaining unit 308 of the first embodiment, except that the received value obtaining unit 308a also obtains the received strength of the radio waves received by the mobile terminal 2a. The received value obtaining unit 308a corresponds to a received strength obtaining unit. Also, the process executed by the received value obtaining unit 308a corresponds to a received strength obtaining process. The radio wave arrival time for the mobile terminal 2a, which is one of the received values, is the radio wave arrival time from the transmission of the impulse signal by the mobile terminal 2a.

The occupant distance identifying unit 311a is similar to the occupant distance identifying unit 311 of the first embodiment, except that the occupant distance identifying unit 311a also identifies the occupant distances from the mobile terminal 2a. The occupant distance identifying unit 311a also corresponds to a distance identifying unit. In addition, the process executed by the occupant distance identifying unit 311a also corresponds to an occupant distance identifying process.

The occupant position estimating unit 312a estimates the position of each of the occupants based on the occupant distances from each of the UWB anchors 320a and the mobile terminal 2a, which are identified by the occupant distance identifying unit 311. The occupant position estimating unit 321a is similar to the occupant position estimating unit 312 of the first embodiment, except that the occupant position estimating unit 321a uses the occupant distance from the mobile terminal 2a to the respective occupants. The process executed by the occupant position estimating unit 312a also corresponds to an occupant position estimating process. Specifically, the position of each of the occupants is estimated by triangulation from the three occupant distances from the UWB anchors 321, 324, and the mobile terminal 2a. In this case, the ranging circle of the mobile terminal 2a may be determined from the position of the mobile terminal 2a relative to the vehicle. The position of the mobile terminal 2a relative to the vehicle may be the terminal position estimated by the terminal position estimating unit 306.

According to the configuration of the second embodiment, the mobile terminal 2a is used as a radar, thereby reducing the number of the UWB anchors 320a in the vehicle. In other words, the number of radars installed in the vehicle can be reduced. The UWB anchor 320a having a function as a radar is more expensive than the UWB anchor 32 without the function as a radar. This is because the UWB anchor 320a with the function as a radar requires an IC that can switch between the transmission and reception of radio waves quickly. In contrast, according to the configuration of the second embodiment, the number of radars installed in the vehicle can be reduced, thereby reducing the cost of the vehicle. In the configuration of the second embodiment, as in the first embodiment, the position of each of the occupants in the vehicle can be estimated based on multiple waveforms for the occupants. Thus, the position of each of the occupants in the vehicle cabin can be estimated more accurately while distinguishing between the occupants.

The correcting unit 313 may correct the distance from the mobile terminal 2a to each of the occupants, which is identified by the occupant distance identifying unit 311a. That is, the correcting unit 313 may correct the occupant distance from the mobile terminal 2a. This is because, depending on the position of the mobile terminal 2a in the vehicle cabin, the occupant distance from the mobile terminal 2a (hereinafter referred to as the terminal occupant distance) is identified as being longer than the true value. The details will be described as follows.

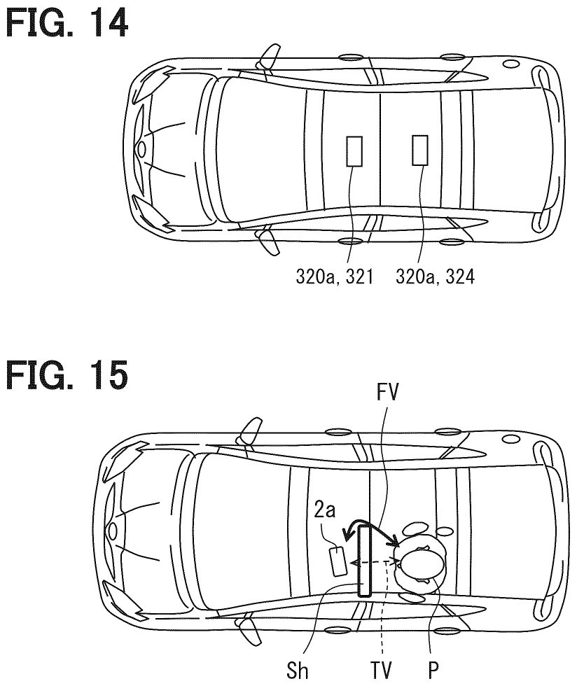

As shown in FIG. 15, when the seat of the vehicle is positioned between the occupant and the mobile terminal 2a, radio waves are shielded by the seat. In this case, the radio waves bypass the seat, so that the radio wave arrival time becomes longer by the amount of time for the radio waves to bypass the seat. Thus, the terminal occupant distance identified by TOF becomes longer than the true value. The reference sign Sh in FIG. 15 indicates the seat. the reference sign P in FIG. 15 indicates the occupant. The reference sign TV in FIG. 15 indicates the true value of the terminal occupant distance. The reference sign FV in FIG. 15 indicates the terminal occupant distance identified by the occupant distance identifying unit 311a.

The longer the distance the radio waves bypass, the lower the received strength of the reflected waves from the occupant (hereinafter referred to as reflected received strength). Thus, even with the same terminal occupant distance, the longer the distance the radio waves bypass, the lower the reflected received strength obtained. Therefore, the terminal occupant distance can be corrected by a difference between the actual value and the ideal value based on a correspondence between the terminal occupant distance and the reflected received strength. The ideal correspondence between the terminal occupant distance and the reflected received strength (hereinafter referred to as the ideal relationship) can be set in advance through simulations or the like. The ideal relationship is the correspondence between the true value of the terminal occupant distance and the reflected received strength in free space. The ideal correspondence may be stored in the non-volatile memory of the communication ECU 30a in advance. The actual correspondence between the terminal occupant distance and the reflected received strength (hereinafter referred to as the actual correspondence) is the correspondence between the terminal occupant distance identified by the occupant distance identifying unit 311a and the received strength of the radio wave at a point on the waveform used to identify the terminal occupant distance. The correcting unit 313 may correct the terminal occupant distance identified by the occupant distance identifying unit 311a to a shorter value as the reflection received strength becomes weaker compared to the ideal correspondence. As an example, the terminal occupant distance is corrected to a shorter value by 3 cm per 1 dB.

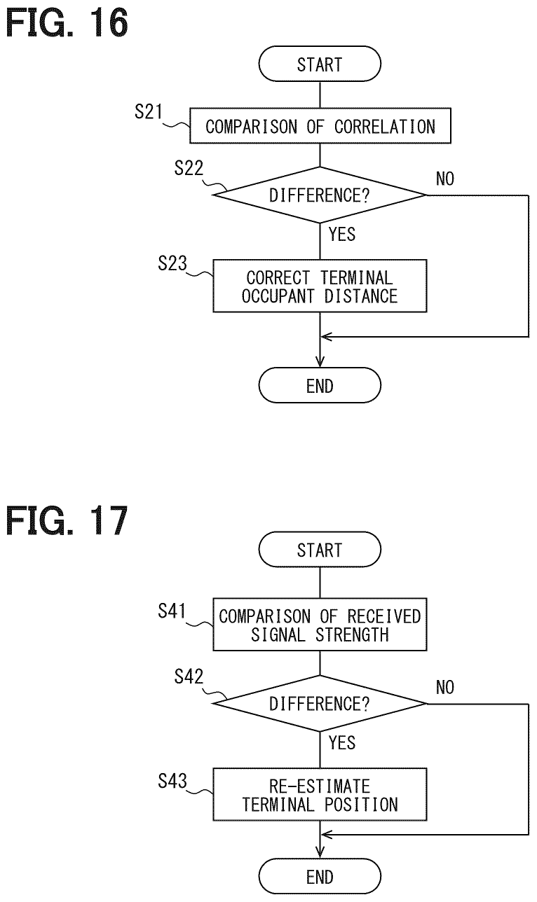

The occupant position estimating unit 312a may estimate the position of each of the occupants using the corrected distance from the mobile terminal 2a by the correcting unit 313 instead of the occupant distance from the mobile terminal 2a identified by the occupant distance identifying unit 311a. With this configuration, it is possible to more accurately estimate the positions of the occupants. In the occupant position estimation process of the second embodiment, the UWB anchor 320 may be replaced with the UWB anchor 320a from that of the first embodiment. In addition, one of the UWB anchors 320 may be replaced with the mobile terminal 2a.

<Distance Correction Process in Communication ECU 30a> Here, with reference to the flowchart in FIG. 16, an example of the process related to the correction of the terminal occupant distance in the communication ECU 30a (hereinafter referred to as distance correction process) will be described. The flowchart in FIG. 16 may be start when the terminal occupant distance is identified by the occupant distance identifying unit 311a.

First, in step S21, the correcting unit 313 compares the reflected received strength in the actual correspondence with the reflected received strength in the ideal correspondence. The occupant distance identifying unit 311a identifies the terminal occupant distance based on a timing on the waveform, and the waveform shows a specific strength value at the timing used to identify the terminal occupant distance. The actual correspondence in this case is the correspondence between the terminal occupant distance identified by the occupant distance identifying unit 311a and the specific strength value at the point on the waveform used to identify the terminal occupant distance.

In step S22, if the correcting unit 313 determines that there is a difference of the reflected received strength between the actual correspondence and the ideal correspondence (YES in S22), the process moves to step S23. On the other hand, if the correcting unit 313 determines that there is no difference (NO in S22), the distance correction process ends. The correcting unit 313 may determine that there is a difference if there is a certain difference of the reflected received strength between the actual correspondence and the ideal correspondence. A certain difference should be considered as a difference greater than a margin of error.

In step S23, the correcting unit 313 corrects the terminal occupant distance to a shorter value as the received strength in the actual correspondence becomes lower compared to that in the ideal correspondence. Then, the distance correction process ends. After the distance correction process ends, the occupant position estimating unit 312a estimates the position of each of the occupants. When the terminal occupant distance is corrected in the distance correction process, the occupant position estimating unit 312a estimates the position of the occupant using the corrected terminal occupant distance. If the terminal occupant distance is not corrected in the distance correction process, the occupant position estimating unit 312a estimates the position of the occupant using the uncorrected terminal occupant distance.

<Terminal Position Re-estimation Process in Communication ECU 30a> The position of the mobile terminal 2a may change during the vehicle moving, which is different from the case of the UWB anchors 320a. Thus, when the position of the mobile terminal 2a changes, it is preferable to re-estimate the terminal position. If the position of the mobile terminal 2a changes, the received strength reflected by the same occupant changes. Therefore, the communication ECU 30a may re-estimate the terminal position according to the change in the reflected received strength as a trigger.

Here, with reference to the flowchart in FIG. 17, an example of the procedure of the process related to re-estimation of the terminal position in the communication ECU 30a (hereinafter referred to as the terminal position re-estimation process) will be described. The flowchart in FIG. 17 may start each time the received value obtaining unit 308a obtains a received value.

First, in step S41, the terminal position estimating unit 306 compares the current received strength obtained from the mobile terminal 2a by the received value obtaining unit 308a with the previous received strength. In step S42, if the terminal position estimating unit 306 determines that there is a difference in the received strength between the current and previous times (YES in S42), the process moves to step S43. On the other hand, if the terminal position estimating unit 306 determines that there is no difference (NO in S42), the terminal position re-estimation process ends. In the terminal position re-estimation process, the terminal position estimating unit 306 may determine that there is a difference if there is a certain level of difference in the received signal strength between this time and the previous time. The certain level of difference is a difference greater than an error.

In step S43, the terminal position estimating unit 306 estimates the terminal position again. Then, the terminal position re-estimation process ends. When estimating the terminal position again, the UWB instruction unit 303 causes the UWB anchors 32 to transmit impulse signals. Then, the terminal distance is identified and the terminal position is estimated in the following steps.

According to the above configuration, even if the position of the mobile terminal 2a changes, the position of each of the occupants can be estimated accurately using the re-estimated mobile terminal 2a. Additionally, the terminal position estimating unit 306 does not re-estimate the occupant position unless the terminal position estimating unit 306 determines that there is a difference in the received strength. Therefore, unnecessary processing can be avoided compared to configurations that periodically re-estimate the position of the terminal. Therefore, while avoiding unnecessary processing, the position of each of the occupants can be estimated accurately using the mobile terminal 2a.

(Third Embodiment) In the second embodiment, the configuration is shown in which one mobile terminal 2a is used as a radar to estimate the position of each of the occupants, but the present disclosure is not necessarily limited to this. For example, two or more mobile terminals 2a may be used as radars to estimate the position of each of the occupants (the third embodiment). The two or more mobile terminals 2a are mobile terminals 2a brought into the vehicle. According to the third embodiment, the more the number of mobile terminals 2a that can be used as radars increases, the more accurately the position of each of the occupants can be estimated.

(Fourth Embodiment) In the aforementioned embodiments, the communication ECUs 30 and 30a perform the processes related to the estimation of the position of each of the occupants, but the present disclosure is not necessarily limited to this. For example, the processing related to the estimation of the position of each of the occupants may be performed by the combination of the communication ECU 30, 30a and other ECUs. Additionally, the processing related to the estimation of the position of each of the occupants may be performed by an ECU other than the communication ECUs 30, 30a.

The present disclosure is not limited to the embodiments described above, and various modifications are possible within the scope of the claims. An embodiment obtained by appropriately combining technical means disclosed in different embodiments is also included in the technical scope of the present disclosure. In addition, the control unit and the method described in the present disclosure may be implemented by a dedicated computer that configures a processor programmed to execute one or a plurality of functions embodied by a computer program. Alternatively, the devices and techniques described in this disclosure may be implemented by a dedicated hardware logic circuit. Alternatively, the device and the method described in the present disclosure may be implemented by one or more special purpose computers configured by a combination of a processor executing a computer program and one or more hardware logic circuits. The computer program may be stored in a computer-readable non-transitory tangible storage medium as an instruction executed by a computer.

1. An occupant position estimating device to estimate a position of each of occupants in a vehicle cabin of a vehicle, the occupant position estimating device comprising:
a transmission control unit configured to control each of radars to transmit radio waves, the radars transmitting and receiving radio waves in the vehicle cabin;
a received strength obtaining unit configured to obtain multiple received strengths of the radio waves that are received by the respective radars;
a separating unit configured to obtain a separated waveform for each of the occupants through a process to isolate components corresponding respectively to the occupants from a waveform which indicates a time variation of the multiple received strengths that are received by the received strength obtaining unit;
a headcount identifying unit configured to identify a number of the occupants in the vehicle cabin from the separated waveform for each of the occupants;
a distance identifying unit configured to identify distances from each of the radars to the respective occupants in accordance with the number of the occupants identified by the headcount identifying unit based on a timing at which a human-specific fluctuation appears in the separated waveform;
an occupant position estimating unit configured to estimate a position of each of the occupants in the vehicle cabin based on the distances from each of the radars to the respective occupants.

2. The occupant position estimating device according to claim 1, wherein
the distance identifying unit configured to identify the distances from each of the radars to the respective occupants based on a timing at which a fluctuation caused by respiration of corresponding one of the occupants appears in the separated waveform.

3. The occupant position estimating device according to claim 1, wherein
the received strength obtaining unit is configured to separately obtain:
reflection characteristic values that are received strengths of the radio waves that are transmitted by one of the radars and received by the one of the radars; and
transmission characteristic values that are received strengths of the radio waves that are transmitted by one of the radars and received by another of the radars,
the separating unit is configured to obtain the separated waveform for each of the occupants through the process to isolate components corresponding respectively to the occupants from a waveform which indicates a time variation of the reflection characteristic values and a waveform which indicates a time variation of the transmission characteristic values, and
the headcount identifying unit is configured to identify the number of the occupants in the vehicle cabin based on the separated waveform for each of the occupants.

4. The occupant position estimating device according to claim 1, wherein
the radars are at least three radars.

5. The occupant position estimating device according to claim 4, wherein
at least one of the at least three radars is a mobile terminal that is carried by one of the occupants,
a position of the mobile terminal relative to the vehicle is identified by a system of the vehicle using communication between the mobile terminal and at least three antennas disposed in the vehicle,
the other of the at least three radars is a radio wave detector that is disposed in the vehicle,
a position of the radio wave detector relative to the vehicle is predetermined, and
the occupant position estimating unit is configured to estimate the position of each of the occupants in the vehicle cabin based on a distance from the position of the mobile terminal, which is identified by the system of the vehicle, to each of the occupants and a distance from the position of the radio wave detector, which is predetermined, to each of the occupants.

6. The occupant position estimating device according to claim 5, wherein
the distance identifying unit identifies the distance from the mobile terminal to the respective occupants based on the timing at which the human-specific fluctuation appears in the separated waveform,
the separated waveform shows a specific strength value at the timing used to identify the distance,
the occupant position estimating device further comprising
a correcting unit configured to correct the distance from the mobile terminal based on the specific strength value and an ideal correlation between a received strength value of the radio waves and a distance from the mobile terminal in a predetermined free space, wherein
the occupant position estimating unit is configured to estimate the position of each of the occupants in the vehicle cabin using the corrected distance from the mobile terminal.

7. An occupant position estimating method executed by at least one processor to estimate a position of each of occupants in a vehicle cabin of a vehicle, the occupant position estimating method comprising:
controlling each of radars to transmit radio waves, the radars transmitting and receiving radio waves in the vehicle cabin;
obtaining multiple received strengths of the radio waves that are received by the respective radars;
obtaining a separated waveform for each of the occupants through a process to isolate components corresponding respectively to the occupants from a waveform which indicates a time variation of the multiple received strengths;
identifying a number of the occupants in the vehicle cabin from the separated waveform for each of the occupants;
identifying distances from each of the radars to the respective occupants in accordance with the identified number of the occupants based on a timing at which a human-specific fluctuation appears in the separated waveform for each of the occupants;
estimating a position of each of the occupants in the vehicle cabin based on the distances from each of the radars to the respective occupants.

8. An occupant position estimating device to estimate a position of each of occupants in a vehicle cabin of a vehicle, the occupant position estimating device comprising:
a transmission control unit configured to control each of radars to transmit transmission waves at predetermined intervals during a uniquely given transmission period that is set to the respective radars, wherein each of the radars receives reflected waves of the transmission waves reflected in the vehicle cabin;
a received strength obtaining unit configured to acquire a plurality of received strength waveforms based on the reflected waves received by the radars, wherein each of the plurality of received strength waveforms indicates a time variation of strength of a corresponding one of the reflected waves received by the radars;
a separating unit configured to isolate separated waveforms for the occupants from composite waveforms which are formed by superimposing the plurality of received strength waveforms;
a headcount identifying unit configured to identify a number of the occupants in the vehicle cabin based on the separated waveforms obtained by the separating unit;
a distance identifying unit configured to calculate distances from each of the radars to the respective occupants in accordance with the number of the occupants identified by the headcount identifying unit based on timings at which fluctuations caused by the occupants appear in the separated waveforms; and
an occupant position estimating unit configured to estimate the position of each of the occupants in the vehicle cabin based on the distances calculated by the distance identifying unit.
